SEQUENCE LISTING (13 sequences)

SEQ ID NO: 1 - DNA, 23 bp, Artificial Sequence
  Description of Artificial Sequence Primer Rsceen1s
gacaggacagtgcttgtttaagg

SEQ ID NO: 2 - DNA, 23 bp, Artificial Sequence
  Description of Artificial Sequence Primer Rscreen1as
tgactacacaatattgctcgcac

SEQ ID NO: 3 - DNA, 13139 bp, Mus musculus
  Description Rosa 26 locus sequence
aagcttctcacgtagcaaccagagctccagagccagcagctgctgccgccttgtatactcactcctgtgatccaacacaggagcaaccttttctttaccccacccccacttcttaacacacttttttttggggggggggggggaacaagtgctccatgctggaaggattggaactatgcttttagaaaggaacaatcctaaggtcacttttaaattgaggtctttgatttgaaaatcaacaaataccaaattccaaatattcgttttaattaaaccagcaatgtggatataagcattaagttttagttttaaaaaggtcaattttccaaacattcagcaatcatatttaaatttacagctaggaacaagagccttgggtcatgtcctaccaaagaacataactcaatattctacacatgacaatctgaataaccttaaagcctctaatcccataacaggccacaaattttggacagagaactaatgatcctcctgagaaaactggaagaaatccagggaaaagaaattcctgtgtcctccaaactcagaaatctctaattatgtcagtattctctgctttagtcctaggtcagattgcacacatctaaaataacctcttaaagttttcctcctagcgacctaaaccattattaatatcaaattaaccatcaaaacactttcctctcaatatgctgcacacaaacctcctcctggaacctcctccatctggatcctccccaatcaaaagtataggtatttaacatataagcaaggaagtaatgtaaacatgaccttggtcacaaatatgtcatctaaaaacaatttagtcaaggtatggaggaaattcgagaacctgaatctttttaagtattttgagcacaggaacaattggcaaaaggaatccaggtatagacaaaacccagagcccagagctctgggcgaaaaatgagttgctggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtcgaatttctaaaagggcgaat

SEQ ID NO: 4 - DNA, 1073 bp, Mus musculus
  Description 5′ arm of Rosa 26
caggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtct

SEQ ID NO: 5 - DNA, 4333 bp, Mus musculus
  Description 3′ arm of Rosa 26
tagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatc

SEQ ID NO: 6 - DNA, 13544 bp, Artificial Sequence
  Description of Artificial Sequence Rosa 26 targeting vector pOMP3  with zsgreen
ctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgactaggggtaaccataacttcgtataatgtatgctatacgaagttatttataatctagaactagtggatccacgattcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcagggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgataacttcgtataatgtatgctatacgaagttatggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgggcaccggtgtttttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctagagtcgcggccgctcagggcaaggcggagccggaggcgatggcgtgctcggtcaggtgccacttctggttcttggcgtcgctgcggtcctcgcgggtcagcttgtgctggatgaagtgccagtcgggcatcttgcggggcacggacttggccttgtacacggtgtcgaactggcagcgcaagcggccaccgtccttcagcagcaggtacatgctcacgtcgcccttcaagatgccctgcttgggcacggggatgatcttctcgcaggagggctcccagttgtcggtcatcttcttcatcacggggccgtcggcggggaagttcacgccgtagaacttggactcgtggtacatgcagttctcctccacgctcacggtgatgtcggcgttgcagatgcacacggcgccgtcctcgaacaggaaggagcggtcccaggtgtagccggcggggcaggagttcttgaagtagtcgacgatgtcctgggggtactcggtgaacacgcggttgccgtacatgaaggcggcggacaagatgtcctcggcgaagggcaaggggccgccctccaccacgcacaggttgatggcctgcttgcccttgaaggggtagccgatgccctcgccggtgatcacgaacttgtggccgtccacgcagccctccatgcggtacttcatggtcatctccttggtcaggccgtgcttggactgggccatggtggcgaccggtggatccactagttctagctgtaggaaaaagaagaaggcatgaacatggttagcagaggctctagggccgccctgggagtggacacctgtggagagaaaggcaaagtggatgtcaatgtcactcaagtgtatggccagatctcaagcctgccacacctcaagcttgacaacaaaaagattgtcttttctgaccagatggacgcggccaccctcaaaggcatcaccgcgggccaggtgaatatcaaatcctcctcgtttttggaaactgacaatcttagcgcagaagtcatgcccgcttttgagagggagtactcaccccaacgcttactagagccgccggtcacacgccagaagccgaaccccgccctgccccgtcccccccgaaggcagccgtccccctgcggcagccccgaggctggagatggagaaggggacggcggcgcggcgacgcacgaaggccctccccgcccatttccttcctgccggcgccgcaccgcttcgcccgcgcccgctagagggggtgcggcggcgcctcccagatttcggctccgccagatttgggacaaaggaagtccctgcgccctctcgcacgattaccataaaaggcaatggctgcggctcgccgcgcctcgacagccgccggcgctccggggccgccgcgcccctcccccgagccctccccggcccgaggcggccccgccccgcccggcacccccacctgccgccaccccccgcccggcacggcgagccccgcgccacgccccgcacggagccccgcacccgaagccgggccgtgctcagcaactcggggaggggggtgcaggggggggttacagcccgaccgccgcgcccacaccccctgctcacccccccacgcacacaccccgcacgcagcctttgttcccctcgcagcccccccgcaccgcggggcaccgcccccggccgcgctcccctcgcgcacacgcggagcgcacaaagccccgcgccgcgcccgcagcgctcacagccgccgggcagcgcgggccgcacgcggcgctccccacgcacacacacacgcacgcaccccccgagccgctcccccccgcacaaagggccctcccggagccctttaaggctttcacgcagccacagaaaagaaacgagccgtcattaaaccaagcgctaattacagcccggaggagaagggccgtcccgcccgctcacctgtgggagtaacgcggtcagtcagagccggggcgggcggcgcgaggcggcgcggagcggggcacggggcgaaggcaacgcagcgactcccgcccgccgcgcgcttcgctttttatagggccgccgccgccgccgcctcgccataaaaggaaactttcggagcgcgccgctctgattggctgccgccgcacctctccgcctcgccccgccccgcccctcgccccgccccgccccgcctggcgcgcgccccccccccccccgcccccatcgctgcacaaaataattaaaaaataaataaatacaaaattgggggtggggaggggggggagatggggagagtgaagcagaacgtggggctcacctcgacccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatagtccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactagtcaataatcaatgtcgaaaacctgcaggcatgcaagctagcccaaacctatattaccctgccctagcgtaactatttaaatggccggcccctgcaggccgcggtggagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacg

SEQ ID NO: 7 - DNA, 12739 bp, Artificial Sequence
  Description of Artificial Sequence Rosa 26 targeting vector pOMP1 with TK
ctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgactaggggtaaccataacttcgtataatgtatgctatacgaagttatttataatctagaactagtggatccacgattcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcagggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgataacttcgtataatgtatgctatacgaagttatggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacg

SEQ ID NO: 8 - DNA, 15342 bp, Artificial Sequence
  Description of Artificial Sequence Rosa 26 locus targeted with targeting vector
aagcttctcacgtagcaaccagagctccagagccagcagctgctgccgccttgtatactcactcctgtgatccaacacaggagcaaccttttctttaccccacccccacttcttaacacacttttttttggggggggggggggaacaagtgctccatgctggaaggattggaactatgcttttagaaaggaacaatcctaaggtcacttttaaattgaggtctttgatttgaaaatcaacaaataccaaattccaaatattcgttttaattaaaccagcaatgtggatataagcattaagttttagttttaaaaaggtcaattttccaaacattcagcaatcatatttaaatttacagctaggaacaagagccttgggtcatgtcctaccaaagaacataactcaatattctacacatgacaatctgaataaccttaaagcctctaatcccataacaggccacaaattttggacagagaactaatgatcctcctgagaaaactggaagaaatccagggaaaagaaattcctgtgtcctccaaactcagaaatctctaattatgtcagtattctctgctttagtcctaggtcagattgcacacatctaaaataacctcttaaagttttcctcctagcgacctaaaccattattaatatcaaattaaccatcaaaacactttcctctcaatatgctgcacacaaacctcctcctggaacctcctccatctggatcctccccaatcaaaagtataggtatttaacatataagcaaggaagtaatgtaaacatgaccttggtcacaaatatgtcatctaaaaacaatttagtcaaggtatggaggaaattcgagaacctgaatctttttaagtattttgagcacaggaacaattggcaaaaggaatccaggtatagacaaaacccagagcccagagctctgggcgaaaaatgagttgctggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgactaggggtaaccataacttcgtataatgtatgctatacgaagttatttataatctagaactagtggatccacgattcgagggcccctgcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcagggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgataacttcgtataatgtatgctatacgaagttatggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtcgaatttctaaaagggcgaat

SEQ ID NO: 9 - DNA, 1823 bp, Artificial Sequence
  Description of Artificial Sequence PGK-neo-pA
ggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcagggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgca

SEQ ID NO: 10 - DNA, 1867 bp, Artificial Sequence
  Description of Artificial Sequence CAGGS- Intron
tcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagtaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacag

SEQ ID NO: 11 - DNA, 703 bp, Artificial Sequence
  Description of Artificial Sequence ZsGreen ORF
cgccaccatggcccagtccaagcacggcctgaccaaggagatgaccatgaagtaccgcatggagggctgcgtggacggccacaagttcgtgatcaccggcgagggcatcggctaccccttcaagggcaagcaggccatcaacctgtgcgtggtggagggcggccccttgcccttcgccgaggacatcttgtccgccgccttcatgtacggcaaccgcgtgttcaccgagtacccccaggacatcgtcgactacttcaagaactcctgccccgccggctacacctgggaccgctccttcctgttcgaggacggcgccgtgtgcatctgcaacgccgacatcaccgtgagcgtggaggagaactgcatgtaccacgagtccaagttctacggcgtgaacttccccgccgacggccccgtgatgaagaagatgaccgacaactgggagccctcctgcgagaagatcatccccgtgcccaagcagggcatcttgaagggcgacgtgagcatgtacctgctgctgaaggacggtggccgcttgcgctgccagttcgacaccgtgtacaaggccaagtccgtgccccgcaagatgcccgactggcacttcatccagcacaagctgacccgcgaggaccgcagcgacgccaagaaccagaagtggcacctgaccgagcacgccatcgcctccggctccgccttgccctga

SEQ ID NO: 12 - DNA, 36 bp, Artificial Sequence
  Description of Artificial Sequence pA
aaataaagcaatagcatcacaaatttcacaaataaa

SEQ ID NO: 13 - DNA, 7078 bp, Artificial Sequence
  Description of Artificial Sequence  vector consisting of a FRT-flanked neomycin resistance gene and a PGK-TK-pA expression cassette
taaagggaacaaaagctgtcgagatctagatatcgatggccatagagttacgctagggataacagggtaatatagccgcggtcgcctaggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcac

(19) United States
(12) Patent Application Publication
(10) Pub. No.: US 2006/0179502 A1
(43) Pub. Date: Aug. 10, 2006

(54) Automated gene-targeting using non-toxic detectable markers

(75) Inventors: Gunther Kauselmann, Koln (DE); Branko Zevnik, Leverkusen (DE); Jost Seibler, Koln (DE); Heidrun Kern, Dusseldorf (DE)

Correspondence Address: NORRIS, MCLAUGHLIN & MARCUS, PA, 875 THIRD AVENUE, 18TH FLOOR, NEW YORK, NY, 10022, US

(73) Assignee: ARTEMIS PHARMACEUTICALS GMBH, KOLN (DE)

(21) Appl. No.: 10/544,683

(22) PCT Filed: Feb. 3, 2004
(86) PCT No.: PCT/EP04/00968

§ 371 Date: Mar. 9, 2006

Related U.S. Application Data
(60) Provisional application No. 60/446,921, filed on Feb. 12, 2003.

(30) Foreign Application Priority Data
Feb. 5, 2003 (EP) 03002424.4

(51) Int. Cl. A01K 67/027; C12N 5/06; C12N 15/87

(52) U.S. Cl. 800/18; 435/455; 435/354

(57) ABSTRACT

The invention relates to a method that enables automated identification and isolation of cells harbouring a predetermined genetic modification (homologous recombination) using detectable/sortable markers, e.g. fluorescence markers, to identify homologous DNA modifications. Suitable vectors are also provided.

The invention relates to a method that enables automated identification and isolation of cells harbouring a predetermined genetic modification (homologous recombination) using detectable/sortable markers, e.g. fluorescence markers, to identify homologous DNA modifications. Suitable vectors are also provided.

### BACKGROUND

Gene targeting in cells, including embryonic stem (ES) cells, relies on the homologous recombination (HR) between a native chromosomal gene and introduced exogenous DNA (Smithies, O. et al., Nature, 317(6034):230-4 (1985); Thomas, K. R. et al., Cell, 44(3):419-28 (1986); Doetschman, T. et al., Proc. Natl. Acad. Sci. USA 85(22):8583-7. (1988); Thomas, K. R., Capecchi, M. R., Cell, 51(3):503-12 (1987)). The method requires transfection of the foreign DNA (targeting vector) into the ES cells, usually by electroporation. HR is a very inefficient process, working in the range of 1×10⁻⁵−1'10⁻⁶ events per electroporated ES cells (for review see Hooper, M. C., Harwood, Embryonal Stem Cell: Introducing Planned Changes into the Animal Geneline, New York (1993)). Most genes of interest are not directly selectable. The identification of homologous recombined (gene targeted) ES cells therefore require the application of selection mechanism.

Positive selection is usually achieved by incorporation of an antibiotic resistance gene into the targeting vector, located between at least 2 stretches of DNA homologous to the targeting region. Upon addition of antibiotic to the culture medium of electroporated ES cells, expression of the positive selection marker allows survival only of those cells which have stably introduced the electroporated DNA into their genome.

Additionally, negative selection strategies further enrich selective growth and identification of HR events. A negative selection marker is added at the 5′ or 3′ end of a homologous DNA stretch (Mansour, S. L. et al., Nature, 336:348-352 (1988); U.S. Pat. Nos. 5,487,992 and 5,464,764). Non-homologous DNA regions outside the homologous regions are not incorporated into the genome if HR has occurred. Current protocols (U.S. Pat. Nos. 5,487,992 and 5,464,765) require the addition of a compound to the culture, medium, to mediate selection against incorporation of the marker, effectively killing ES cells, which have undergone random-recombination. Negative selection generally achieves enrichments of only 2-5fold (Sedivy, J. M., Dutriaux, A., Trends Genet., 15(3):88-790 (1999)).

Given optimal vector design to allow homologous recombination, the described selection strategies known in the art (e.g. the positive-negative selection strategies disclosed in U.S. Pat. Nos. 5,487,992 and 5,464,765) allow identification of HR in about 5% of all analysed ES cells. Said selection strategies are certainly not optimal and it is therefore desirable to
(a) optimize enrichment of homologous recombinants,
(b) reduce variability in efficiency of the currently available methods for enrichment,
(c) avoid unwanted potential toxicity of agents for negative or negative and positive selection agents. Gancyclovir, the most widely used agent for negative selection, inhibits DNA polymerase and therefore cellular proliferation (Matthews, T. and Boehme, R., Reviews of Infectious Diseases, 10(3):490-4 (1998)). Such inhibition may not be tolerable under GMP standards, in particular in potential applications of gene targeting for gene therapy in humans.

Moreover, the selection procedures known in the art requires ES cell culture over a period of 7-8 days, to allow selection to work, and clonal expansion of cells to an absolute number of ca. 2000, to enable molecular analysis and further expansion of the cell population. However, culture of ES cells requires adherent growth of the cells on defined substrate, usually mitotically inactivated “feeder cells”. This, in turn, necessitates laborious, manual “picking” of identified ES clones from their substrate to enable individual clonal growth in culture vessels for maintenance and molecular analysis. It was therefore also desirable to reduce the time period required to isolate potential HR ES clones as well as the manual labour, i.e. picking of ES cell clones, i.e. by increasing-the automation of the gene targeting process employing current protocols.

Fluorescent probes on the other side are known as a powerful tool for identification of molecular events insingle cells. The most widely known is the green fluorescent protein (GFP) from bioluminescent jellyfish Aequorea victoria. However, approximately 30 distinct fluorescent proteins have been discovered and cloned from a variety of species (Labas, Y. A. et al., PNAS, 99(7):4256-4261 (2002)), (Matz,. M. V. et al., Nat. Biotechnol., 17(10):906-18 (1999)) for review see: (Zhang, J. et al., Nat. Rev. Mol. Cell. Biol., 3(12):906-18(2002)).

For certain reasons such as the sensitivity of the cells to be transfected, optically detectable markers including fluorescent probes such as GFP and its variants were only seldom employed as detectable markers in gene targeting experiments. E. g. WO 02/06630 discloses that GFP might be utilized instead of a neomycin phosphotransferase gene in a gene targeting process similar to the basic positive selection protocol. Furthermore WO 03/0022725 discloses that fluorescent gene cassettes can be used as an alternative to negative selection markers in order to further enrich ES cell clones which underwent homologous recombination.

A key property of murine ES cells is that they can be maintained indefinitely in vitro if cultured in the presence of the cytokine leukemia inhibitory factor (LIF) (Smith, A. G. and Hooper, M., Dev. Biol. 121:1-9 (1987); Williams, R. L. et al. .(1988)). ES cells retain the capacity to participate normally in embryogenesis and contribute to all tissues of the mouse embryo when introduced into host blastocysts, including the germ line (reviewed by Robertson, E. J., Oxford, UK, IRL Press: 71-112.(1937); Smith; A. G., Sem. Cell. Biol. 3:385-399 (1992)). However, retention of germ-line transmission competence is often elusive. It depends absolutely on adherence to a rigorous tissue culture regime, with avoidance of any untoward selective pressures. Furthermore, ES cells are only modestly able to amplify if plated as single cells to form clonal populations. The plating efficiency is reported to range from 3 to 5% (Reid, L. H. et al., Mol. Cell. Biol. 11: 2767-2777 (1991); Templeton, N. S., et al., Gene Therapy 4(7): 700-9 (1997)).

Altogether, low plating efficiency, differentiation accompanied by loss of germ line-competence and subsequent failure to produce genetically modified mice is still the key obstacle for the employment of automated sorting methodology for ES cells in gene targeting studies.

Methods such as Fluorescence Activated Cell Sorting have been described for sorting of human (Schuldiner, M. J. et. al., Stem Cells 21(3):257-265 (2003)) and murine (Reddy, S. et al., Proc. Natl. Acad. Sci. USA 89:6721-6725 (1992); WO 03/002272) ES cells, however such cells were only bulk sorted as pools of marker expressing cells for subsequent analysis.

High frequency gene targeting and isolation of single cells with defined characteristics in an automated fashion as described in this application is of great advantage to improve effectiveness and precision of gene targeting experiments.

### SUMMARY OF THE INVENTION

It was now found that certain vectors for targeted homologous recombination which contain one or more expression cassettes coding for a detectable marker and being placed outside the targeting cassette (i.e. the region of homology of the vector to the genomic DNA) allow rapid and reliable distinction, preferably visual distinction, between targeted and non-targeted ES cells in an automated fashion. One example, of such an optically detectable marker is ZsGreen (Clontech, Palo Alto, Calif.). A particular example is the application of a first fluorescence marker gene (e.g. ZsGreen) inside the region of homology (i.e. inside the targeting cassette) and a second, fluorescence marker gene differing from the first marker gene outside the region of homology. Moreover, it was found that with a gene targeting vector containing a positive selection marker (e.g. neomycin) inside the targeting cassette and a fluorescent marker outside the region of homology (e.g. ZsGreen) identification of both (stably transfected) non-targeted and correctly targeted ES cells via optical detection was possible.

Furthermore, stably gene targeting vector transfected ES cells have been automatically analyzed by fluorescence (Cytocon™300; Evotec Technologies) and plated as single cells (Cytocon™ Single Cell Fraction Collector; Evotec Technologies). Both high resolution analysis and single cell plating can be combined in Image Activated Cell Sorting (Elektra, Evotec Technologies). Amplification of clonally plated ES cells enriched for gene targeting events allowed for the first time the isolation of gene targeting events without need for any manual picking or sub-cloning of cells. Furthermore, the contact free procedure and gentle cell handling kept targeted ES cells germline-competent and allowed the production of chimeric and, by tetraploid complementation (Nagy, A. et al., Proc. Natl. Acad. Sci. USA 90-8424-8428 (1993); Eggan, K. et al., Proc. Natl; Acad. Sci., USA 98(11):6209-14 (2001)) fully ES cell derived animals.

Altogether, this method allows for the first time automated gene-targeting from transfection of the gene targeting vector to establishment of totipotent targeted ES clones.

The invention thus provides

(1) a method for isolation of primary mammalian after homologous recombination comprising
(a) transfecting starting primary cells with a homologous targeting vector comprising
   (A) a homologous targeting cassette which comprises (i) a functional DNA segment and a positive selection marker or a functional DNA segment and a first detectable marker, and (ii) two DNA segments homologous to the integration site within the genome of the primary mammalian cells flanking (i); and
   (B) an expression cassette harboring a DNA sequence coding for a second detectable marker different from said first detectable marker, said expression cassette (B) being connected with said homologous targeting cassette (A) so as to allow distinction between targeted and non-targeted cells;
(b) manual or automatic identification and/or isolation of cells containing the positive selection marker or the first detectable marker; and
(c) manual or automatic identification and/or isolation, of cells as homologous recombinants by the absence of the second detectable marker;
(2) a method for preparing transgenic tissues, organs and/or multi-cell organisms which comprises utilizing the method as defined in (1) above; and
(3) a vector for targeted homologous recombination of eukaryotic cells as defined in (1) above.

The vector utilized in the homologous recombination process of (1) above allows direct or indirect distinction between correctly targeted and non-targeted cells. Indirect distinction can be achieved via addition of non-toxic compounds to the cells or via metabolic or enzymatic action of the gene product (detectable marker) encoded by the expression cassette.

By utilizing one or more fluorescence markers as detectable markers, the method (1) of the invention
a) allows to optically distinguish random DNA integrants (light emission) and HR eukaryotic cells, including ES cells (no light emission) (FIG. 2);
b) enhances isolation and reduces variability of recovery of HR ES cells in individual experiments since the addition of compounds to the culture medium is avoided;
c) allows selective, automated distinction of growing transfected ES cell colonies by plating of ES cells, transformed with such light emission targeting vectors, in a way such that preferentially single cell clones grow within a spatially separated region (1 well of a multiwell-dish) and subsequent fluorescence analysis (such ES clones can then automatically be expanded and taken to molecular analysis (FIG. 2));
d) allows selective, automated sorting of growing transfected ES cells for the absence of the first detectable marker and/or the presence of the second detectable marker and/or by Image Activated Cell Sorting (i.e. fluorescence).

The method of the invention has the following advantages over current technology:
(i) The detection strategy allows selective identification of gene targeted ES clones without exposure of ES cells to potentially toxic chemicals, protein or mutagens usually used for negative selection;
(ii) automated optical selection of clones enriched for homologous recombination event by applying gentle sorting machinery equipment such as but not limited to Cytocon™300 & Cytocon™ Single Cell Fraction Collector (Evotec Technologies) or Elektra (Evotec Technologies);
(iii) delivery of isolated single cells in 1 well of a multiwell dish which retain the capability of clonal growth;
(iv) identification of a higher percentage of clones showing homologous recombination compared to standard procedures (i.e. positive-negative selection, positive-positive selection) due to specific Promoter and selectable marker configurations (WO 03/002725);
(v) automated expansion, isolation and molecular analysis of gene targeted ES clones with proven characteristics of totipotency as demonstrated by the generation of chimeric and tetraploid mice;
(vi) significant reduction of both manual labour and associated costs for gene targeting experiments.

### SHORT DESCRIPTION OF THE FIGURES

FIG. 1: Selection mechanism in homologous recombination: A: targeting vector; B: homologous genomic DNA; C: homologous recombined DNA, presence of first marker, absence of second marker.

FIG. 2: Scheme of manual and automated gene targeting process employing novel detectable markers (solid arrows indicate experiments described in this application).

FIG. 3: Generation of targeted ES cells. Scheme of the gene targeting strategy. Both constructs, (i) the targeting vector for positive and negative selection (pOMP1) (3A), and (ii) the targeting vector for positive and optical detection (pOMP3) (3B) were used for insertion of the neo gene into the Rosa26 locus by homologous recombination, as depicted. E: EcoRV; X: XbaI.

(3C) Southern blot analysis of genomic DNA from ES cells transfected with constructs described in (FIG. 4A) and (FIG. 4B). The DNA of ES cells was digested with EcoRV and hybridized with probe 1 specific for the rosa26 locus. The sizes of the wt and the targeted allele are 11.5 kb and 2.5 kb, respectively. The picture shows the analysis of identified non-fluorescent clones.

(3D) Southern blot analysis of genomic DNA from ES cells transfected with pOMP3 and automatically sorted and plated using Cytocon™300.

FIG. 4: Growth of targeted fluorescent and non-fluorescent (marked with arrow) ES clones.

FIG. 5: Enrichment of gene targeting frequency in non-fluorescent ES clones.

FIG. 6 shows schematically the functional segments of the rosa26 locus.

FIG. 7 shows the vector pOMP3.

FIG. 8 shows the vector POMP1.

FIG. 9 shows schematically the modified rosa26 locus.

FIG. 10 shows schematically the CAGGS promoter followed by an intron.

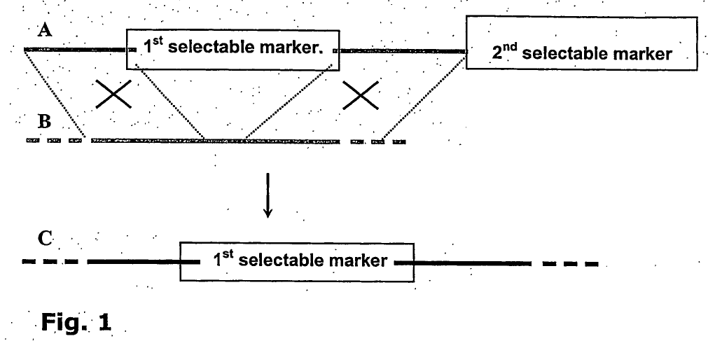

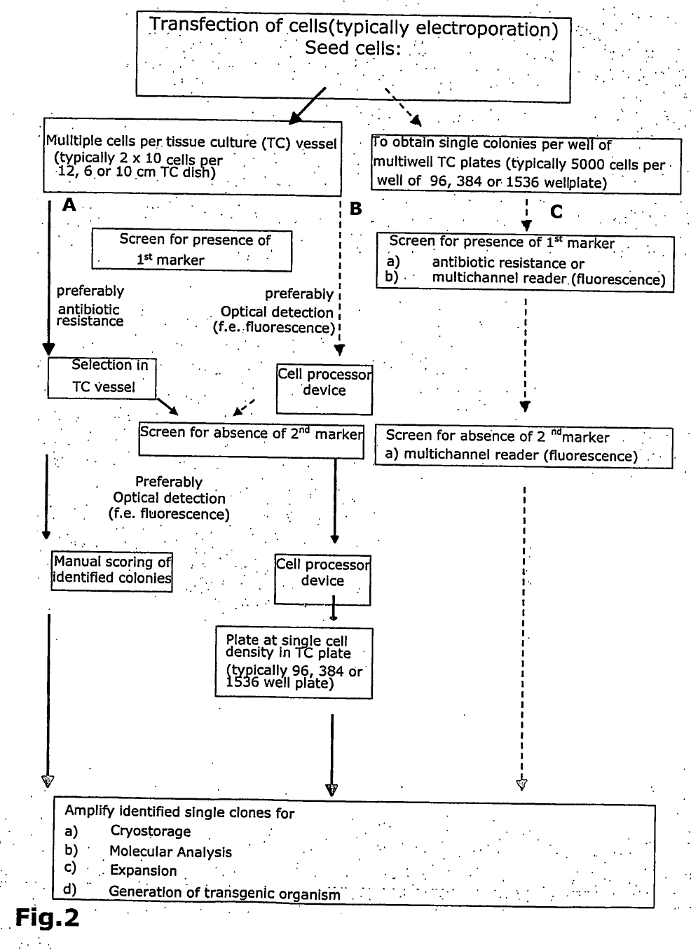

### DETAILED DESCRIPTION OF THE INVENTION

“Targeted vector” or “vector for targeted homologous recombination” according to the present invention is a DNA construct that contains sequences “homologous” to endogenous chromosomal nucleic acid sequences which flank a desired genetic modification. The flanking homologous sequences direct the targeting vector to the specific chromosomal location within a genome by virtue of the homology that exists between the flanking homologous sequences and the corresponding endogenous sequences and introduce the desired genetic modification (i.e. a functional DNA segment) by a process referred to as “homologous recombination”.

“Homologous” means two, or more nucleic acid sequences that are either identical or similar enough that they are able to hybridize to each other or undergo intermolecular exchange.

A “flanking DNA” is a segment of DNA that is collinear with and adjacent to a particular point of reference.

“Gene targeting” is the modification of an endogenous chromosomal locus by the insertion into, deletion of or replacement of the endogenous sequence via homologous recombination using a targeting vector.

The “homologous targeting cassette” is that part of the targeted vector that carries the desired genetic modification, the flanking homologous sequences and optionally functional sequences such as selectable or detectable markers.

The “expression cassette” is that part of the targeted vector that enables distinction of non-targeted and correctly targeted stably transfected cells by determining its presence or absence in the transfected cellular system. The two cassettes are “connected” through covalent linkage of an arbitrary number of nucleotides.

A “detectable marker” only allows for the detection and subsequent isolation of a transfected cell, whereas a “selectable marker” or “selection marker” provides for a selection advantage of the cell containing said marker over cells devoid of said marker and therewith allows for the enrichment of rare transfected cell expressing the marker from the majority of treated cells in the population.

“Multi-cell organisms” according to the present invention refers to non-human mammalians. The “transgenic tissues” or “transgenic organs” are derived from the multi-cell organisms referred to above.

A “non-human organism” or “non-human mammal” is an organism or mammal that is not normally accepted by the public as being human.

As set forth above, the vector used for targeting the primary mammalian cells in the method of (1) above has the following properties: The homologous targeting cassette (A) comprises (i) a functional DNA segment and a positive selection marker or a functional DNA segment and a first-detectable marker, and (ii) two DNA segments homologous to the integration site within the genome of the primary mammalian cells flanking said functional DNA segment and said positive selection marker or said functional DNA segment and said first detectable marker. The expression cassette (B) comprises a DNA-sequence coding for a second detectable marker different from said first detectable marker.

It is preferred that the expression cassette (B) comprises a gene coding for said first detectable marker under the control of a promoter (e.g. constitutive, inducible, etc.) active in primary mammalian cells. Preferably the promoter is a constitutive promoter including, but not limited to, CAGGS (Niwa, H. K. et al., Gene 108(2):193-9 (1991)), CMV (Bi, J. X. et al., Biotechnol. Bioeng. 81(7):848-54 (2003)), c-fos (Bi, J. X. et al., Biotechnol. Bioeng. 81(7):848-54 (2000)), PGK (Adra, C. N. et al., Gene 60:65-74 (1987)), SV40 (Southern, P.J., Berg, P., J. Mol. Appl. Genet. 1:327-341 (1982)), elongation factor 1alpha (eF1alpha), RNA polymerase II (Soriano, P. et al., J. Virol. 65(5):2314-9 (1991)) and TK.

Moreover, it is preferred that said first and/or second detectable marker are a non-toxic, directly or indirectly detectable compound or the marker(s) are gene(s) expressing membrane bound protein(s) or protein(s) with membrane anchoring signal sequence, requiring that such proteins are not expressed in the transfected cell of interest. Such membrane bound proteins can be identified by binding to fluorescence or otherwise labeled detectable antibodies.

Preferably the detectable marker is a compound which is detectable by calorimetric (Lowry method, Bradford reaction (Comassie blue), sulforhodamine B, β-galactosidase (lacZ), placental alkaline phosphatase (PAP)), “fluorescence, bioluminescence such as luciferases from phengodid beetles, which can produce green to red bioluminescence. Viviani, V. R., Cell Mol. Life Sci. 59(11):1833-50 (2002)) phosphorescence detection methods or the like (for review of probes see (Haughland, R. P., Molecular Probes Handbook of fluorescent probes and research chemicals, 172-180, 221-229 (1992-1994); Freshney, R. I., Culture of animal cells, 3^{rd} ed., Wiley & Sons, Inc. (1994)). Most preferred is the use of a fluorescence protein/peptide.

In another preferred embodiment, the first and/or second detectable marker, preferably the second detectable marker is a membrane bonded protein selected from CD1a, CD2, CD3, CD4, CD5, CD6, CD8, CD11, CD14, CD15, CD16, CD19, CD20, CD22, CD25, CD27, CD30, CD33, CD34, CD43, CD45, CD56, CD61, CD62, CD69, CD71, CD90, CD105, CD117, CD123, CD133, CD138, BDCA-2, BDCA-3, BDCA-4, CRTH2, ErbB-2, Ep-CAM (Human Epithelial Antigen HEA), etc. Particularly preferred is that the positive selection marker is neomycine and the second selectable marker is selected from ZsGreen CD4 and CD8, or the first and second selectable markers are ZsGreen and HcRed.

According to the invention the expression cassette may be positioned 5′ or 3′ of a homologous targeting cassette. If the homologous targeting cassette harbors at least two regions of target gene homology which differ in length, the position of the expression cassette is preferably connected with the longer region of homology.

According to the invention the primary mammalian cells include pluripotent and totipotent cells and preferably are embryoinic stem cells (hereinafter shortly referred to as “ES cells”) (excluding human ES cells if this is not patentable). Preferably the primary mammalian cells are derived from a rodent, such as mouse, rat, etc. Particularly preferred are mouse ES cells.

In a more preferred embodiment the vector of (1) above the functional DNA segment of the targeting cassette is a DNA sequence encoding a gene of interest, or a part of a gene of interest, or is a functional DNA sequence which can be converted into such DNA sequence encoding a gene of interest, or intronic sequence, or is a regulatory functional DNA sequence, including, but not limited to, splice receptor of splice donor or acceptor sequences, and recombinase recognition site(s) (RRS(s)).

The functional DNA segment may further comprise gene expression control elements including, but not limited to, ubiquitous or tissue specific promoter, either constitutive or inducible, a polyadenylation signal, intron sequences, recombinase recognition site(s), enhancer recognition site(s), and matrix attachment region(s) (MAR).

The positive selection marker according to the present invention may be a DNA sequence encoding a protein conferring resistance against cell poison, including, but not limited to, neomycine (Beck, E. et al., Gene 19(3):327-36 (1982)), hygromycine (Bernard, H. U. et al., Exp. Cell Res. 158(1):237-43 (1985); Gordano, T. J.; McAllister, W. T., Gene 88(2):285-8-(1990); Santerre, R. F. et al., Gene 30(1-3):147-56 (1984)), puromycine N actyltransferase (puro) (de la Luna, S et al., Gene 62(1):121-6 (1988); de la Luna, S., Ortin, J., Methods Enymol. 216:376-85 (1992)), E. coli xanthine guanosine phosphoribolsyltransferase (gpt) (Spring, K. J. et al., Biochim. Biophys. Acta 1218(2):158-62 (1994)), blasticidin (Kobayashi, K. et al., Agric. Biol. Chem. 55(12):3155-7 (1991); Kimura, M. et al., Mol. Gen. Genet. 242(2):121-9 (1994)), hypoxanthine-guanosine phosphorybosyltransferase (Hprt) (Albertini, R. J. et al., Nature 316(6026):369-71 (1985); Lester, S. C. et al., Somatic Cell. Genet. 6(2):241-59 (1980)), herpes simplex virus type 1 thymidine kinase (hsvTK) (Borrelli, E. et al., Proc. Natl. Acad. Sci. USA 85(20):7572-6 (1988)), and adenine phosphoribosyltransferase (aprt) (Lester, S. C. et al., Somatic Cell. Genet. 6(2):241-59 (1980))—HPRT, TK and aprt in positive selection schemes require ES cells with mutations in their endogeneous Hprt or TK loci—or may be a DNA sequence, conferring superior metabolic properties to the cells, as compared to non-transfected (starting) cells, including the utilization of xanthine, adenine, etc.

According to the present invention the first and/or second marker are DNA sequences encoding a protein allowing direct optical detection, such as by detection of fluorescence, chemiluminescence or the like, or indirect optical detection, such as a colorimetric assay or the like, provided that said second detectable marker differs from said first detectable marker in order to allow separate detection. It is particularly preferred that the first, and/or second detectable marker peptide is a fluorescence protein including, but not limited to, green fluorescence protein (GFP) and homologous, analogous or color variants thereof, and Reef Coral Fluorescent, Proteins (RCFPs) such as AmCyan, ZsGreen, ZsYellow, DsRed, AsRed and HcRed (Clontech,, Palo Alto, Calif.). Particularly preferred are the fluorescent peptides mentioned in the following Table 1 (Labas, Y. A. et al., PNAS 99(7):4256-4261 (2002)):

TABLE 1
Summary of family of GFP-like proteins

| | Original | | |
|---|---|---|---|
| Protein ID | ID | GenBank accession # | Genus species |
| amajGFP | amFP486 | AF 168421 | Anemonia majano |
| dsfrGFP | DsFP483 | AF 168420 | Discosoma striata |
| clavGFP | CFP484 | AF 168424 | Clavularia sp. |
| GFP | | M 62653 | Aequora victoria |
| cgigGFP | | AY 037776 | Condylactis gigantea |
| hcriGFP | | AF 420592 | Heteractis crispa |
| ptilGFP | | AY 015995 | Ptilosarcus sp. |
| rmueGFP | | AY 015996 | Renilla muelleri |
| zoanGFP | zFP560 | AF 168422 | Zoanthus sp. |
| asulGFP | asFP499 | AF 322221 | Anemonia sulcata |
| dis3GFP | | AF 420593 | Discosoma sp.3 |
| dendGFP | | AF 420591 | Dendronephthya sp. |
| mcavGFP | | AY 037769 | Montastraea cavernosa |
| rfloGFP | | AY 037772 | Ricordea florida |
| scubGFP1 | | AY 037767 | Scolymia cubensis |
| scubGFP2 | | AY 037771 | Scolymia cubensis |
| zoanYFP | | AF 168423 | Zoanthus sp. |
| DsRed | drFP583 | AF 168419 | Discosoma sp.1 |
| dis2RFP | dsFP593 | AF 272711 | Discosoma sp.2 |
| zoan2RFP | | AY 059642 | Zoanthus sp.2 |
| mcavRFP | | AY 037770 | Montastraea cavernosa |
| rfloRFP | | AY 037773 | Ricordea florida |
| asulCP | asCP | AF 246709 | Anemonia sulcata |
| hcriCP | hcCP | AF 363776 | Heteracis crispa |
| cgigCP | cpCP | AF 363775 | Condylactis gigantea |
| cpasCP | cpCP | AF 383155 | Condylactis parsiflora |
| gtenCP | gtCP | AF 383156 | Goniopora tenuidens |

It is moreover preferred that the positive selection marker or the second detectable marker are flanked by RRSs in order to allow removal of the marker by means of a recombinase after the proper recombination product was established. The flanking homologous DNA segments may have a length of 0.1 to 20 kb, preferably 0.5 to 10 kb. The actual flanking DNA sequences depend on the locus of integration, suitable sequences are those homologous to the Rosa26, HPRT, beta-actin, GAPDH locus or the like of eukaryotic cells.

In a particularly preferred vector of the invention the first detectable marker is ZsGreen and the constitutive promoter is the CAGGS promoter. The targeting cassette in such a vector may contain a positive selection marker as defined above or a second fluorescence marker differing from the first marker. Suitable marker combinations out of the ones mentioned in Table I are known in the art. Reef Coral Fluorescent Proteins (RCFPs) such as AmCyan, ZsGreen, ZsYellow, DsRed, AsRed and HcRed (Clontech, Palo Alto, Calif.) each emit a distinct wavelength. The corresponding cDNAs were isolated from nonbioluminescent reef corals (class Anthozoa), and have, been optimized for bright emission, fast chromophore maturation, and codon optimized for increased expression in mammalian cells. Because of their distinct spectra RCFPs can be used in combination to visualize multiple events simultaneously.

In a most preferred vector the homologous sequences of the targeting cassette are homologous to the mouse Rosa26 locus and the selection marker is a loxP flanked neomycin resistance gene (see bp 1287 to 3233 of SEQ ID NO:6), preferably said vector has the sequence of SEQ ID. NO. 6.

The method (1) of the invention comprises, but is not limited to, the following steps:
1. Generation of a gene targeting construct harbouring one or mrore expression cassettes coding for non-identical optical markers, e.g. two fluorescence molecules (ZsGreen, AmCyan1 or ZsYellow1 or DsRed2, DsRedExpress or AsRed2 or HcRed1; Clonteich, Palo Alto, Calif.) under control of a constitutive CAGGS promoter (Niwa, H. et al., Gene, 108(2):193-9 (1991)).
2. Gene targeting by homologous recombination in embryonic stem (ES) cells with this vector.
3. Identification of preferentially homologous recombined ES clones harbouring the second detectable marker, not harbouring the first detectable marker.
4. Isolation of such clones by
(a) manual picking of non-fluorescent clones under fluorescence light emission source;
(b) limited dilution plating of electroporated ES cells in multi-well cell culture dishes at a pre-determined concentration to enable preferential growth of single clones/well;
   (i) manual sub-culture and molecular analysis of identified non-fluorescent colonies by current protocols;
   (ii) automated sub-culture and molecular analysis of identified non-fluorescent colonies;
(c) automated cell sorting, preferably by image activated cell sorting of preferentially homologous recombined ES cells expressing or not expressing the detectable fluorescent marker and plating of sorted cells at single cell density in multi-well tissue culture plates.

Said automated sorting is preferably performed with one of the following devices A to C with the particular settings indicated:

(A) Cytocon™ 300 (Evotec Technologies) Equipped with Cytocon™ Single Cell Fraction Collector (Evotec Technologies). Parameters are Preferably as Follows:

I) Software: Cytocon™ software “SWITCH”
II) Cytocon™ 300 sorter chip mounted on an Olympus IX-50 fluorescence microscope equipped with a 100 W lamp and fluorescein filter set (BP510-550, DMS570, BA590). Single cells are preferably caged within the field cage using 5 V at 800 kHz.
III) Cytocon™ Fluidics system connected to the Cytocon™ 300 sorter chip, consisting of
   a) precision syringe pump
      a rate pump1: 1:1000-1500 pl/s
      rate pump 2: −488 pl/s
   b) Fluidic block
   c) Injector
      a. Hamilton syringe, 5-10 μl injection volume
I) Cytocon™ chip driver, settings as follows:
   a. Mode Zero: 605
   b. Mode One: 4.88

Detached fluorescent & nonfluorescent ES cells are preferably resuspended in an isotonic buffered solution (Cytocon-bufferII™) at a density of 1×10⁵ cells/500 μl. Preferably for reduction of electrolytes buffering is achieved with but not limited to carbohydrates such as glucose or insitol. Exposure to such buffer is preferably minimized to less than 30 minute more preferably less than 20 minutes. Fluorescence is assessed by eye (Olympus IX-50 fluorescence microscope equipped with a 100 W lamp and fluorescein filter set (BP510-550, DM570, BA590); objective 10×/0.30 Ph1 UplanF1).

Single cells showing no fluorescence are flushed out of the Sorter Chip with ES cell culture medium and deposited with aid of Cytocon™ Single Cell Fraction Collector (Evotec Technologies) into separate wells of a 96 well microtiter plate pre-filled with ES cell culture medium with the following parameters: Sheath flow: 800000 pl/s (0.8 μl/s)

(B) Elektra (Evotec Technologies)

Sorter chip mounted on an Olympus IX-50 fluorescence microscope equipped with a 100 W lamp and fluorescein filter set (BP510-550, DM570, BA590). Single cells are preferably caged within the field cage using 6V, 700 kHz. Fluidics system connected to the sorter chip, consisting of precision syringe pump (rates pump1: 1:1000-1500 pl/s, pump2: −488 pl/s). Injector with Hamilton syringe (10 μl injection volume)

Detached fluorescent & nonfluorescent ES cells are preferably resuspended in an isotonic buffered solution (Cytocon-bufferII™) at density of 1×10⁵ cells/500 μl.

Preferably for reduction of electrolytes buffering is achieved with but not limited to carbohydrates such as glucose or insitol. Exposure to such buffer is preferably minimized topless than 30. minutes, more preferably less than 20 minutes. Fluorescence is assessed by eye (Olympus IX-50 fluorescence microscope equipped with a 100 W lamp and fluorescein filter set (BP510-550, DM570, BA590); objective 10×/0.30 Ph1 UplanF1).

Single cells showing no fluorescence are flushed out of the Sorter Chip with ES cell culture medium and deposited inrto separate wells of a 96 well microtiter plate pre-filled with ES cell culture medium with the following parameters: Sheath flow: 800000 pl/s (0.8 μl/s)

(C) Macgnetic Bead Separation is Preferably Performed with the BD™ IMag Cell

Separation System (BD Bioscience), more preferably with the MACS® Technology (Milteny Biotec) based on the use of MACS MicroBeads, MACS Columns and MACS Separators; or separation is performed with the CliniMACS® Cell Selection System.

The method (2) of the invention for preparing transgenic tissues, organs and/or multi-cell organisms (except for human being or human tissues and organs, if such are not patentable under the respective regulations) is exemplified by the following steps:
(a) Generation of genetically modified organisms by
   (aa) ES cells. Such cells can be used to create genetically modified rats or mice by standard blastocyst injection technology (Robertson, E. J. Editor., IRL Press: Oxford, UK, 71-112 (1987)), aggregation (Wood, S. A. et al., Nature 365(64411):87-89 (1993)), tetraploid blastocyst injection (Wang, Z. Q. et al., Mech. Dev. 62(2):137-45 (1997)), nuclear transfer and cloning (Wakayama, T. et al., Proc. Natl. Acad. Sci. USA 96(26):14984-9 (1999)), ES cells derived from other organism such as rabbits (Wang, Z. Q. et al., Mech. Dev. 62(2):137-45 (1997); Schoonjans, L. et. al., Mol. Reprod. Dev. 45(4):439-43 (1996)), chickens (Pain, B. et al., Development 122(8):2339-48 (1996)), or other species;
   (bb) modified protoplasts to generate genetically modified plants;
   (cc) nuclear transfer from modified eukaryotic cells to oocytes to generate cloned organisms with modified allele (Wakayama, T. et al., Proc. Natl. Acad. Sci. USA 96(26):14984-9 (1999); Baguisi, A. et al., Nat. Biotechnol. 17(5):456-61 (1999); Wilmuti I. L. et al., Nature 385(6619):810-3.(1997); Wilmut, I. L. et al., Reprod. Fertil. Dev. 10(7-8):639-43 (1998); Wakayama, T., Protein, Nucleic Acid Enzyme 45(13 Suppl.):2005-14 (2000); Wakayama, T. et al., Nature 394(6691):369-74 -(1998); Rideaut, W. M., Nat. Genet. 24(2):109-10(2000); Campbell, K. H. et al., Nature, 380(6569):64-6 (1996));
   (dd) cell-fusion to transfer the modified allele to another cell, including transfer of engineered chromosome(s), and uses of such cell(s) to generate organisms carrying, the modified allele or engineered chromosome(s) (Kuroiwa, Y. et al., Nat. Biotechnol. 18(10):1086-90 (2000)).
(b) Cloning: The techniques used to construct DNA vectors described herein are standard molecular biology techniques well known to the skilled molecular biologist (Sambrook, J. Fritsch, E. F. a.M., Molecular Cloning: a Laboratory Maunal, 2^{nd} ed., vols. 1, 2 and 3, Cold Spring Harbor, N.Y., USA, Wiley Interscience, NY (1989)).
(c) Gene modifications including
   (aa) deletion of coding sequences, gene segments, or regulatory elements;
   (bb) alterations(s) of coding sequence, gene segments, or regulatory elements including substitutions, additions, and fusions (e.g. epitope tags or creation of bifunctional proteins);
   (cc) insertion of new coding regions, gene segments, or regulatory elements, such as those for selectable marker genes or reporter genes or putting new genes under endogenous transcriptional control;
   (dd) creation of conditional alleles, e.g. by introduction of loxP sites flanking the region to be excised by Cre recombinase (Abremski, K., Hoess, R., J. Biol. Chem. 259(3):1509-14 (1984)) or FRT sites flanking the region to be excised by FLP recombinase (Andrews, B. J. et al., Cell 40(4):795-803 (1985); Meyer-Leon, L. et al., Cold Spring Harb. Symp. Quant. Biol. 49:797-804 (1984)); or
   (ee) replacement of coding sequences or gene segments from one species with orthologous coding sequences from a different species, e.g. replacing a murine genetic locus with the orthologous human genetic locus to engineer a mouse where that particular locus has been ‘humanized’.
   (d) Introduction of vectors into eukaryotic cells: Using standard methodology, such as transfection mediated by calcium phosphate, lipids, or electroporation (Sambrook, J. Fritsch, E. F. a.M., Molecular Cloning: a Laboratory Maunal, 2^{nd} ed., vols. 1, 2 and 3, Cold Spring Harbor, N.Y., USA, Wiley Interscience, NY (1989)).
   (e) Selection: By exposure to selection agents, depending on the selectable marker gene that has been engineered into the vector. If the selectable marker is the neomycin phosphotransferase (neo), gene (Beck, E. et al., Gene 19(3):327-36 (1982)) then cells that have taken up the vector can be selected in G418-containing media; cells that do not have the vector will die, whereas cells that have taken up the vector will survive (Freshney, R. I., Culture of animal cells, 3^{rd} ed., Wiley & Sons, Inc. (1994)). Other suitable selectable markers include any drug that has activity in eukaryotic cells, such as hygromycin B Santerre, R. F. et al., Gene 30(1-3):147-56 (1984); Bernard, H. U. et al., Exp. Cell. Res. 158(1):237-43); Giordano, T. J., McAllister, W. T., Gene 88(2):285-8 (1990)), Blasticidin (Izumi, M. et al., Exp. Cell. Res. 197(2):229-33 (1991)) and other which are familiar to those skilled in the art.

The invention is further described by reference to the following examples, which is, however, not to be construed so as to limit the invention.

### EXAMPLE

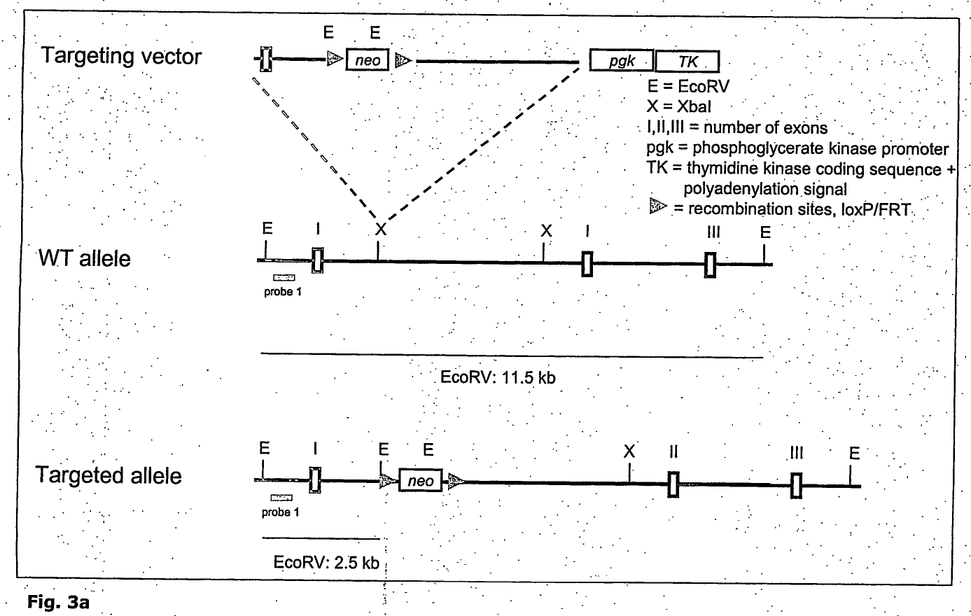

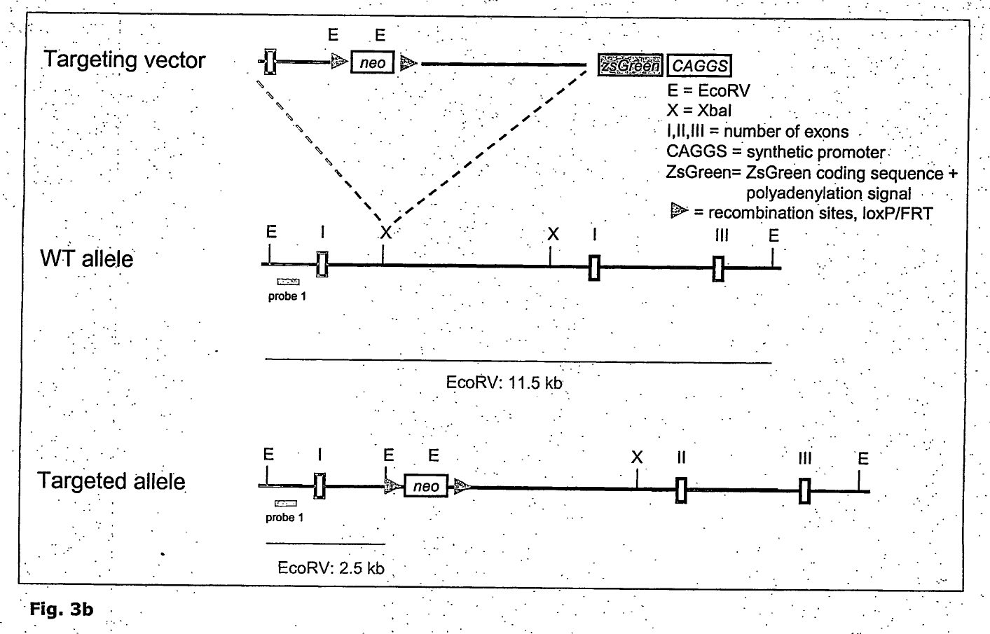

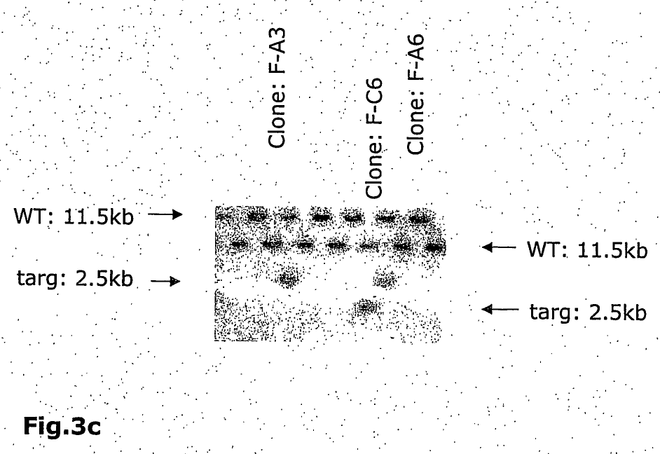

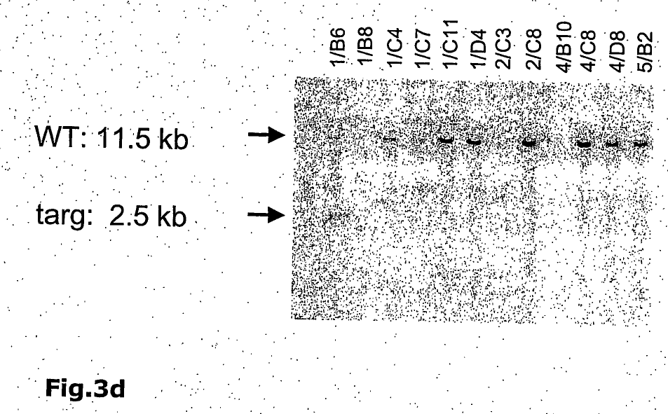

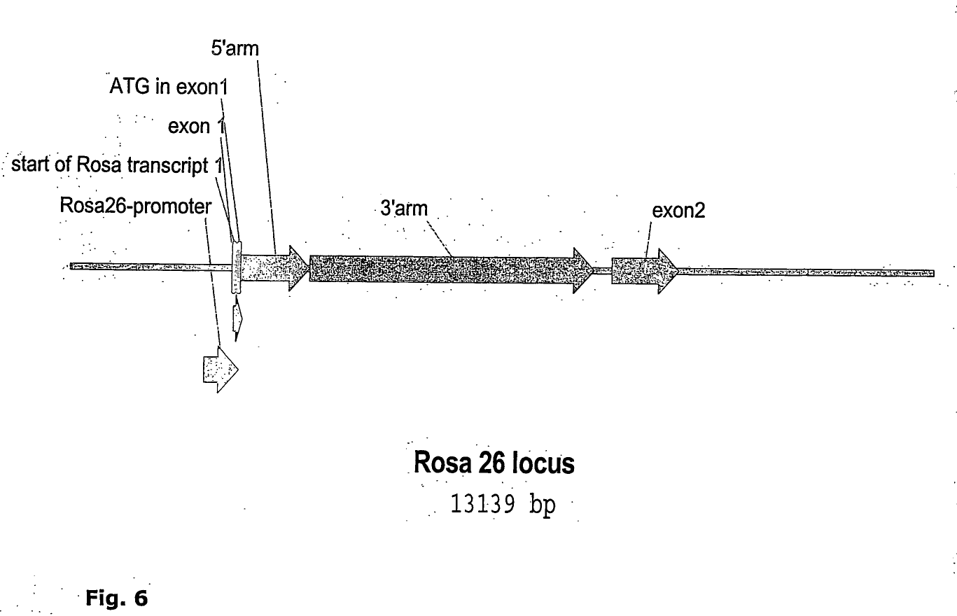

As example for optical identification of ES clones, the fluorescent molecule ZsGreen gene (Clontech) was chosen. The Rosa locus of the mouse (FIG. 6, SEQ ID NO:3) was chosen for homologous recombination. The targeting strategy is outlined in FIGS. 3A, B and C and D.

1. Rosa Targeting Vector: A 129 Sv/Ev-BAC library (Incyte Genomics) was screened with a probe against exon2 of the Rosa26 locus (amplified from mouse genomic DNA using Rscreen1s (GACAGGACAGTGCTTGTlTTAAGG; SEQ ID NO:1) and Rscreen1as (TGACTACACAATATTGCTCGCAC; SEQ ID NO:2)). PCR conditions were as follows: 95° C., 2 min, followed by 30 cycles: 95° C. 30 s; 60° C., 30 s; 72° C, 30 s; 72° C., 7 min; followed by 20° C., 2 min. Out of the identified BAC clone a 11 kb EcoRV subfragment was inserted into the HindII site of pBS. Two fragments, a 1 kb SacII/XbaI fragmenit (SEQ ID NO:4) and a 4 kb XbaI-fragment (SEQ ID NO:5) were used as homology arms and inserted into a vector consisting of a FRT-flanked neormycin resistance gene and a PGK-TK-pA expression cassette for negative selection (SEQ ID NO: 13)

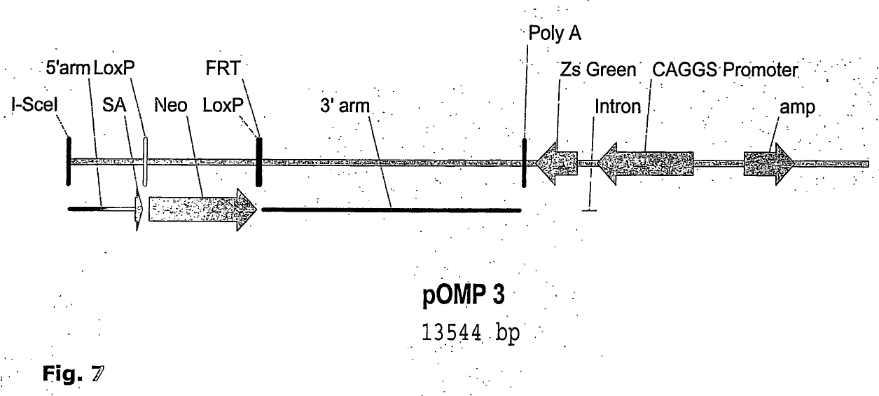

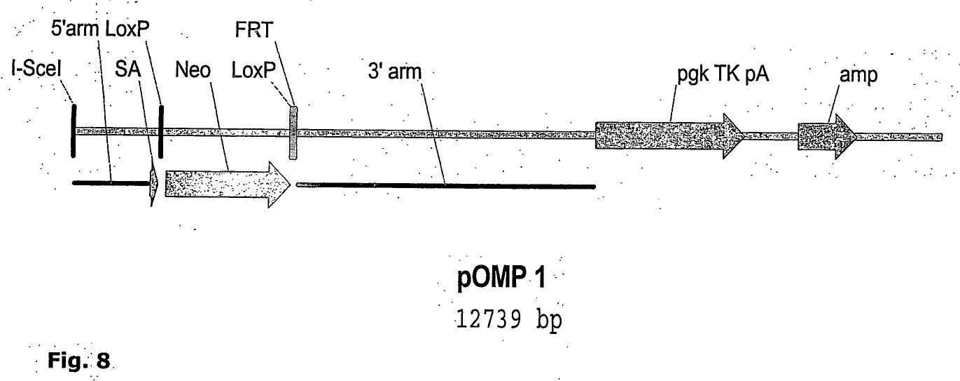

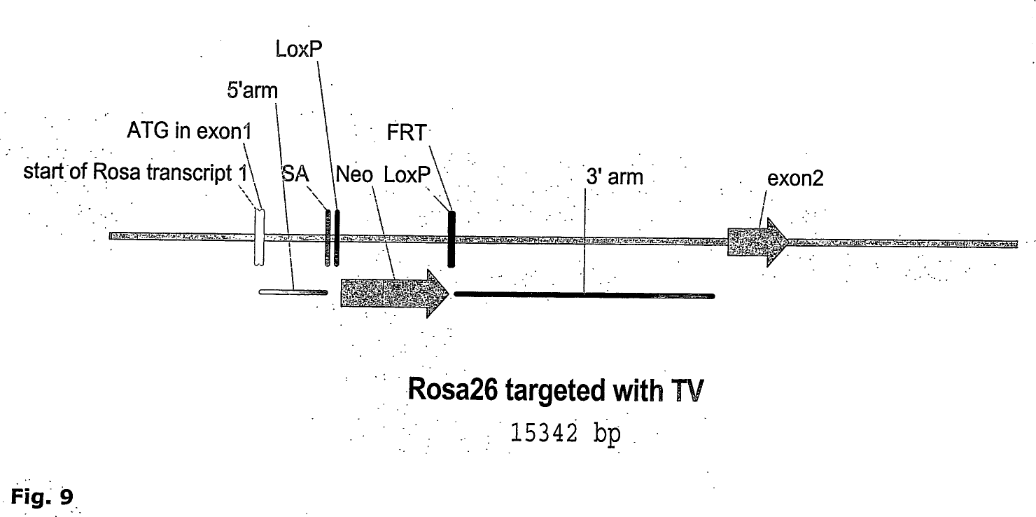

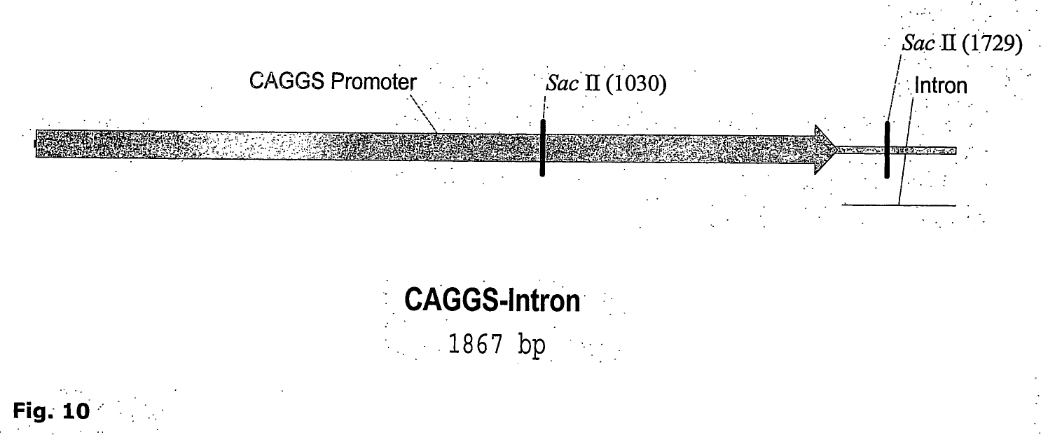

2. pOMP1 and pOMP3 vector Construction: For construction of pOMP1 (FIG. 8 and SEQ ID NO:7), the FRT-flanked neomycin resistance gene was replaced by a loxP-flanked neomycin resistance gene (SEQ ID NO:9). The ZsGreen gene was placed under the control of the CAGGS-promoter (SEQ ID NO:11) followed by a synthetic intron (pMultilink CAGGS-zsGreen; FIG. 10, SEQ ID. NO:10). This expression cassette was used for a replacement of PGK-TK-pA of pOMP1 resulting in pOMP3 (FIG. 7 and SEQ ID NO:6). pOMP1, and pOMP3 were used for the targeting experiments (FIGS. 3A, 3B). For ES cell electroporation, vectors were linearized at the I-SceI restriction site.

3. Introduction of pOMP vectors into ES cells

The ES cell line Art4.12 (Eggan, K. et al., Nat. Biotechnol., 20(5):p. 455-9 (2002)) was grown on mitotically inactivated feeder layer (Mitomycin C (Sigma M-0503)) comprised of mouse embryonic fibroblasts in medium composed of 1× DMEM high Glucose (Invitrogen 41965-062), 4 mM Glutamin (Invitrogeen 25030-024), 1× Non Essential Amino Acids (Invitrogen 11140-035) 0.1 mM Sodium Pyruvat (Invitrogen 11360-039), 20 mM Hepes (Invitrogen 15630-056), 0.1 mM β-Mercaptoethanol (Invitrogen 31350-010); 2×10⁶ μ/l Leukemia Inhibitory Factor (Chemicon ESG 1107) and 15% fetal bovine serum (PAN 1302-P220821).

Linearized vectors POMP1 and pPOMP 3 were introduced into the cells by electroporation using a Gene Pulser with Capacitance Extender (Biorad).

Rapidly growing cells were used on the first day following the last passage. Upon trypsinization (Invitrogen 25200-056) cells were resuspended in PBS (Invitrogen 20012-019) and preplated for 25 min on gelatinized 10 cm plates to remove unwanted feeder cells. The supernatant was harvested, ES cells were washed once in PBS and counted (Neubauer hemocytometer). 10⁷ cells were mixed with 30 μg of I-Sce linearized vector in 800 μl of transfection buffer (20 mM Hepes; 137 mM NaCl, 15 mM KCI, 0.7 mM Na₂HPO₄, 6 mM Glucose 0.1 mM β-Mercaptoethanol in H₂O) and electroporated using a Biorad Gene Pulser with Capacitance Extender set on 250 V and 500 μF. Electroporated cells were seeded at a density 0.25×10⁷ cells per 10 cm tissue culture dish onto a previously prepared layer of neomycin-resistant inaictivated mouse embryonic fibroblasts (MEF). For automated selection of non-fluorescent clones (experiment 6), cells were seeded on 6 cm gelatine-coated tissue culture dishes without MEF layer.

4. Manual identification of ES cells containing a targeted disruption of the Rosa locus

48 h after electroporation, the medium was replaced on all dishes by medium containing 250 μg/ml Geneticin (Invitrogen 10131-019) for positive selection of G418 resistant ES clones.

5 day after electroporation, ½ of the dishes containing ES cells electroporated with vector pOMP1 were negatively selected by addition of 2 uM Gancyclovir (Cymeven®, Roche)

On day 8 after electroporation ES colonies were isolated as follows:

Medium was replaced by PBS and the culture dishes were placed on the stage of a binocular, (Nikon SMZ-2B). Using low magnification (25×) individual ES clones of undifferentiated appearance were removed from the surface of the culture dish by suction into the tip of a 20 ul pipette (Eppendorf). The harvested clones were placed in individual wells of 96 well plates containing 30 ll of 2.5% Trypsin (Invitrogen). After disruption of clones by pipetting with a multichannel pipette (Eppendorf), cells were seeded onto feeder containing 96 well plates with pre-equilibrated complete ES-medium.

For pOMP1 positive selected clones (Geneticin) and positive/negative selected clones (Geneticin and Gancyclovir) were harvested as described above.

For pOMP3 only positive selected clones were isolated either at random as described above, or the ES clones were placed under a binocular (Leica MZFL III) with fluorescent light source (GFP3 Filters set) and bright-field illumination. At dim light, clearly identifiable non-fluorescent ES clones (FIG. 4) were isolated as described above.

Cells were grown for 3 days with daily medium changes and then splitted 1:2 on gelatinized (Sigma G-1890) 96 well plates. 3 days after splitting cells were lysed, genomic DNA was prepared and analysed by Southern blot.

5. Identification of non-fluorescent transfected cells by plate-reader analysis

Electroporated cells were seeded at a density of 1 cell per well in a 96 multi-well plate (Nunc, Order #136101), coated with a previously prepared layer of neomycin-resistant inactivated mouse embryonic fibroblasts.

48 h after electroporation, the medium was replaced on all dishes by medium containing 250 μg/ml Geneticin (Invitrogen 10131-019) for positive selection of G418 resistant ES clones.

5 days upon transfection individual wells were scored by plate-reader analysis

a) by O. D. measurement for the presence of single ES clones
b) by fluorescence measurement (GFP3 filter set; Leica, Bensheim, Germany) for the location of non-fluorescent clones.
   Identified non-fluorescent ES clones were expanded and taken to molecular analysis by Southern blotting (see FIGS. 3A, 3B) according to standard procedures.

6. Image Activated Cell Sorting for the Analysis of Transfected Cells and Single Cell Plating

48 h after electroporation, the medium was replaced on all dishes by medium containing 250 μg/ml Geneticin (Invitrogen 10131-019) for positive selection of G418 resistant ES clones.

On day 7 or 8 after electroporation ES colonies were automatically sorted by Cytocon™ 300. (Evotec Technologies) based on the presence or absence of fluorescent light emission of single clones. Non-fluorescent ES cells were automatically plated employing a Cytocon™ Single Cell Fraction Collector (Evotec Technologies) in single entities of a 96-well plate.

Transfected ES cells were enzymatically removed from by trypsin addition (Invitrogen 25200-056). After centrifugation, cells were resuspended in Cytocon buffer II at a density of 1×10⁵ cells/500 μl. Five to ten μl of 1:5 in buffer II diluted cells were applied to Cytocon™ 300 using a Hamilton syringe. Sorting and single cell plating were undertaken with the following. Cytocon™ 300 settings: Generator modes 0 605; 1 4.88; pump rates pump1: 1:1000-1500 pl/s; pump 2: −488 pl/s; “sheat flow”: 800000 pl/s (0.8 μl/s). A Cytocon™ Sorter Chip was mounted on a fluorescent microscope and single cells were caged within the field cage. Fluorescence was assessed by eze and non-fluorescent cells were automatically seeded with the aid of the Cytocon™ Single Cell Fraction Collector (Evotec Technologies), into single entities (individual well of 96-well tissue culture plate (Greiner Cellstar), coated with MEF cells and pre-equilibrated complete ES-medium).

Clones were grown for 7 days with one medium change and then replated and cultured 1:1 onto a second 96-well-tissue culture plate. Clones were subsequently expanded, aliquots cryoconserved and genomic DNA prepared and molecularly analysed by Southern blot.

7. Injection of ES Cells into Diploid and Tetraploid Blastocysts and Generation of Chimeric and ES Mice

Embryo culture was carried out in microdrops on standard bacterial petri dishes (Falcon) under mineral oil (Sigma). Modified CZB media (Chatot et al, Supra (?)) was used for embryo culture unless otherwise noted. Hepes buffered CZB was used for room temperature operations.

Chimeras were generated as described (Hogan, B., Beddington, R., Costantini, F. & Lacy, E. eds. Manipulating the Mouse Embryo, a Laboratory Manual. 2^{nd} ed. Cold Spring, Harbor, N.Y.: Cold Spring Harbor, Laboratory. Press, 1994). Briefly, Balb/C host embryos were harvested at dpc 3.5 from the uterus of superovulated Balb/c OlaHsd females (Harlan, Netherlands) mated with Balb/c OlaHsd males (Harlan Netherlands). For microinjection, 5-6 blastocysts were placed in a drop of DMEM with 15% FCS under mineral oil. A flat tip, piezo actuated microinjection-pipette with an internal diameter of 12-15 μm was used to inject 20-30 ES cells into each blastocyst. After recovery, 8 injected blastocysts were transferred to each uterine horn of 2.5 days post coitum, pseudopregnant NMRI females that had been mated with vasectomized males. Litters were controlled, and pups alive by that time were counted as surviving pups. ES cell contribution was judged by coat color chimerism.

Production of mice by tetraploid embryo complementation has been previously described (Eggan et al, PNAS 98:6209-6214 (2001)). After administration of hormones, superovulated B6D2F1females were mated with B6D2F1 males. Fertilized zygotes were isolated from the oviduct and any remaining cumulus cells were removed with hyluronidase. After overnight culture, two-cell embryos were electrofused to produce one cell tetraploid embryos using a CF150-B cell fusion instrument from BLS (Budapest, Hungary) according to the manufacturers instructions. Embryos that had not undergone membrane fusion within 1 hour were discarded. Embryos were then cultured in vitro to the blastocyst stage. For microinjection, 5-6 blastocysts were placed in a drop of DMEM with 15% FCS under mineral oil. A flat tip, piezo actuated microinjection-pipette with an internal diameter of 12-15 μm was used to inject 15 ES cells into each blastocyst. After recovery, ten injected blastocysts were transferred to each uterine horn of 2.5 days post coitum, pseudopregnant NMRI females that had been mated with vasectomized males. Litters were controlled, and pups alive by that time were counted as surviving pups.

8. Results
(a) Effect of fluorescence on ES cell growth

Expression of ZsGreen (pOMP3) did not negatively influence ES cell colony formation. The mean number of resistant ES colonies per 1×10⁷ ES cells transfected with the ZsGreen expression vector pOMP3 is similar to the number of ES colonies transfected with pOMP1, (see Table 2).

TABLE 2
Fluorescence does not negatively effect clonal ES cell growth

| | | | # clones per |
|---|---|---|---|
| | 2^{nd}marker gene | 2^{nd} Selection | 1 × 10⁷ transfected cells |
| pOMP1 | None | none | 3500 |
| POMP1 | Thymidine Kinase | Gancyclovir | 950 |
| pOMP3 | ZsGreen | none | 3366 |

(b) Selective isolation of non-fluorescent ES colonies.

15.5% of all colonies in the pOMP3 gene targeting experiment were non-fluorescent. Isolation of non-fluorescent ES colonies under fluorescent light-source as described above, led to significant enrichment of such ES colonies. By this method 99.3% (all but 1 clone) were non-fluorescent when monitored upon isolation see Table (3).

TABLE 3
Enrichment of isolation of non-fluorescent ES colonies in pOMP3
experiments.

| | | Isolated (%) |
|---|---|---|
| Random isolation | Fluorescent | 84.5 |
| Random isolation | Non-fluorescent | 15.5 |
| Selective isolation | Fluorescent | 0.7 |
| Selective isolation | Non-fluorescent | 99.3 |

(c) HR frequency in pOMP targeting experiments

The analysis of non-fluorescent ES cells for HR events in the case of pOMP3 targeting resulted in a targeting frequency of 6.49% +/−0.5% in 3 independent experiments (Table 4).

This is a 8 fold higher frequency if compared to targeting of pOMP1 without counter-selection. Compared to pOMP1 gene targeting plus counterselection with Gancyclovir (3.02% HR), optical isolation was 2.2 fold more efficient.

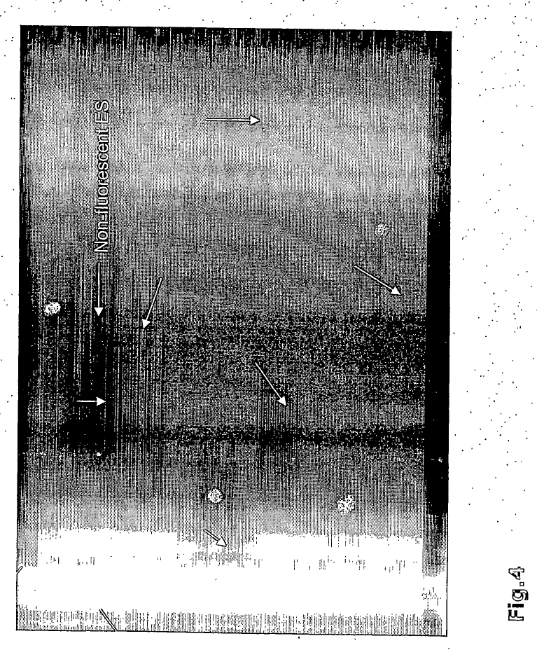

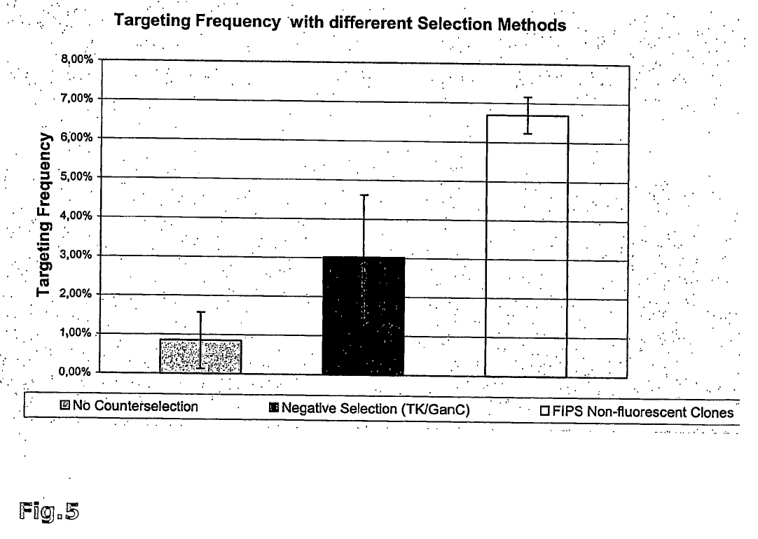

TABLE 4
Enrichment of gene targeting frequency in non-fluorescent
ES clones. (see also FIG. 5)

| | | | HR frequency |
|---|---|---|---|
| Construct | Selection | HR - clones/analyzed | (%) |
| pOMP1 | None | 6/708 | 0.85 |
| pOMP1 | TK/GanC | 10/331 | 3.02 |
| POMP3 | fluorescent | 0/452 | 0.00 |
| POMP3 | Non-fluorescent | 16/239 | 6.69 |

(d) HR frequency in Automated pOMP targeting experiments

Transfected, sorted and individually seeded cells attached and divided at a frequency of 34%. Transfected, sorted and individually seeded cells attached and divided at a frequency of 34%. 9 individual clones were analyzed and HR clones identified at a frequency of 4%.

TABLE 5
Frequency of gene targeting in FACS sorted and single-cell
plated ES clones.

| | Single cell plating | HR | HR |
|---|---|---|---|
| Construct | efficiency | clones/analyzed | frequency |
| POMP3 | 34% | 1/25 | 4% |

(e) The automated gene targeting procedure does not affect competence of mouse production of wild-type “Art4.12” ES cells and HR ES clone “OMP 3 Cyto 1-B6”. Cytocon™ 300 sorted. ES cells produce highly coat colour chimeric mice (see Table 6), and, via tetraploid complementation, solely ES clone derived mice (see Table 7).

TABLE 6
Production of Chimeric mice by diploid Injection.

| | Art 4.12 | Targeted ES clone |
|---|---|---|
| | Parental | OMP 3 Cyto 1-B6 |
| # Injected diploid | 61 | 52 |
| # Pups born | 14 | 13 |
| # Chimeric pups | 11 | 13 |

TABLE 7
Production of ES mice by tetraploid Injection.

| | Art 4.12 | Targeted ES clone |
|---|---|---|
| | Parental | OMP 3 Cyto 1-B6 |
| # Injected tetraploid | 153 | 60 |
| # ES mice born | 1 | 3 |

1. A method for isolation of primary mammalian cells after homologous recombination, said method comprising the following steps:
(a) transfecting starting primary cells with a homologous targeting vector comprising:
(A) a homologous targeting cassette which comprises (i) a functional DNA segment and a positive selection marker or a functional DNA segment and a first detectable marker, arid (ii) two DNA segments homologous to the integration site within the genome of the primary mammalian cells flanking (i); and
(B) an expression cassette harboring a DNA sequence coding for a second detectable marker different from said first detectable marker, said expression cassette (B) being connected with said homologous targeting cassette (A) so as to allow distinction between targeted and non-targeted cells;
(b) manually or automatically identifying and/or isolating cells containing the positive selection marker or the first detectable marker; and
(c) manually or automatically identifying and/or isolating cells as homologous recombinants by the absence of the second detectable marker.

2. The method of claim 1, wherein
(i) the expression cassette (B) comprises a gene coding for said second detectable marker under the control of a promoter active in primary mammalian cells; and/or
(ii) said first and/or second detectable marker is a non-toxic, directly or indirectly detectable compound, is a membrane bound or protein with membrane anchoring signal sequence; and/or
(iii) the expression cassette (B) is positioned 5′ or 3′ relative to the homologous targeting cassette; and/or
(iv) the primary mammalian cells are pluripotent or totipotent cells; and/or
(v) the primary mammalian cells are derived from a rodent.

3. The method of claim 1, wherein
(i) the functional DNA segment of the targeting cassette (A) is a DNA sequence encoding a gene of interest, or is a functional DNA sequence which can be converted into such DNA sequence encoding a gene of interest, or intronic sequence, or is a regulatory functional DNA sequence; and/or
(ii) the functional DNA segment may further comprise gene expression control elements; and/or
(iii) the positive selection marker is a DNA sequence encoding a protein conferring resistance against cell poison, is a DNA sequence, conferring superior metabolic properties to the cells; and/or
(iv) the first and/or second detectable marker are DNA sequences encoding a protein allowing direct optical detection, or indirect optical detection, provided that said second detectable, marker differs from said first detectable marker in order to allow separate detection; and/or
(v) the second detectable marker is a membrane bound or protein with membrane anchoring signal sequence; and/or
(vi) the positive selection marker and the first detectable marker are flanked by one or more RRSs; and/or
(vii) the flanking homologous DNA segments have a length of 0.1 to 20 kb; and/or
(viii) the flanking DNA sequences are homologous to the Rosa26, HPRT, beta-actin, GAPDH locus of the eukaryotic cells.

4. The method of claim 2, wherein
(i) the first and/or second detectable marker peptide is a fluorescence protein, and the constitutive promoter of the expression cassette is selected from GAGGS, CMV, PGK, and TK; and/or
(ii) the first and/or second detectable marker is a protein.

5. The vector of claim 4, wherein the homologous sequences are homologous to the mouse Rosa26 locus.

6. The method according to claim 1, wherein the identification or isolation is performed automatically.

7. The method according to claim 1, which further comprises:
(i) plating of transfected cells into standard tissue culture vessels; and/or
(ii) limited dilution plating of transfected cells into multiwell plates, in order to allow preferential growth and analysis of single clones in individual wells/plate, and optionally
(iii) subsequent detection, and isolation of transfected cells plated by manually or automatically selecting for the absence of the first detectable marker and/or the presence of the second detectable marker.

8. The method of claim 7 wherein the presence of the first and/or absence of the second marker is analyzed by (FACS) mechanism, whereby the isolated sorted cells are re-plated.

9. The method of claim 1, which is for preparing transgenic tissues, organs and/or multi-cell organisms.

10. The method of claim 9, wherein the transgenic multi-cell organism is a non-human mammal, and said method comprises modifying an ES cell.

11. The method of claim 10 which further comprises one or more of the steps:
(i) injecting isolated ES cells in or aggregating isolated ES cells with diploid and/or multiploid preimplantation embryos, or
(ii) injecting in/fusion of nuclei from ES cells with enucleated non-fertilized eggs (nuclear transfer); and/or
(iii) generating transgenic non-human embryos and/or animals.

12. A vector for targeted homologous recombination of eukaryotic cells as defined in claim 1.

13. The method of claim 2, wherein
(i) the promoter active in primary mammalian cells is a constitutive promoter; and/or
(ii) the detectable compound is a compound being detectable by calorimetric, fluorescence, chemiluminescence, or phosphorescence detection methods, or is a membrane bound or protein with membrane anchoring signal sequence, or is a fluorescent peptide; and/or
(iii) the primary mammalian cells are ES cells; and/or
(iv) the primary mammalian cells are mouse ES cells.

14. The method of claim 3, wherein
(i) the functional DNA segment of the targeting cassette (A) is a splice receptor of splice donor or acceptor sequences, and recombinase recognition site(s); and/or
(ii) the functional DNA segment further comprises ubiquitous or tissue specific promoter, either constitutive or inducible, a polyadenylation signal, intron sequences; recombinase recognition site(s) (RRS), enhancer recognition site(s), and/or matrix attachment region(s) (MAR); and/or
(iii) the positive selection marker is a DNA sequence encoding a protein conferring resistance against cell poison, wherein the protein is neomycine, hygromycine, puromycine, histidinol or bleomycine, is a DNA sequence, conferring superior utilization of xanthine or adenine; and/or
(iv) the first and/or second detectable marker are DNA sequences encoding a protein allowing direct optical detection by detection of fluorescence or chemiluminescence, or indirect optical detection by colorimetric assay; and/or
(v) the flanking homologous DNA segments have a length of 0.5 to 10 kb.

15. The method of claim 4, wherein
(i) the first and/or second detectable marker peptide is ZsGreen and the constitutive promoter is the CAGGS promoter; and/or
(ii) the first and/or second detectable marker is selected from CD1a, CD2, CD3, CD4, CD5, CD6, CD8, CD11, CD14, CD15, CD16, CD19, CD20, CD22, CD25, CD27, CD30, CD33, CD34, CD43, CD45, CD56, CD61, CD62, CD69, CD71, CD90, CD105, CD117, CD123, CD133, CD138, BDCA-2, BDCA-3, BDCA-4, CRTH2, ErbB-2, and Ep-CAM (Human Epithelial Antigen HEA), and/or
(iii) the positive selection marker is neomycine and the second selectable marker is selected from ZsGreen CD4 and CD8; and/or
(iv) the first and second selectable markers are ZsGreen and HcRed.

16. The vector of claim 5, wherein said vector has the sequence of SEQ ID. NO.6.

17. The method according to claim 6, wherein the identification or isolation is performed automatically,
(i) the isolation comprises fluorescence activated cell sorting (FACS); and/or
(Ii) the isolation buffer is an isotonic buffered solution; and/or
(iii) the cells to be sorted remain in the buffer for a time shorter than 30 minutes.

18. The method of claim 10, wherein the transgenic multi-cell organism is a mouse, and said method comprises modifying an ES cell.

19. The method of claim 11, which comprises injecting isolated ES cells in or aggregating isolated ES cells with blastocytes.
